(19) United States
(12) Patent Application Publication
(10) Pub. No.: US 2015/0305891 A1
(43) Pub. Date: Oct. 29, 2015

(54) ORTHOPEDIC GUIDE SYSTEMS AND METHODS

(71) Applicant: SMITH & NEPHEW, INC., Memphis, TN (US)

(72) Inventors: Alisha W. Bergin, Southaven, MS (US); Nathaniel M. Quinn, Arlington, TN (US); Jackie Carter-Horne, Memphis, TN (US)

(21) Appl. No.: 14/649,531

(22) PCT Filed: Dec. 5, 2013
(86) PCT No.: PCT/US2013/073309

§ 371 Date: Jun. 3, 2015

Related U.S. Application Data
(60) Provisional application No. 61/733,737, filed on Dec. 5, 2012.

(51) Int. Cl. A61F 2/46; A61B 17/17

(52) U.S. Cl. CPC A61F 2/4609; A61B 17/1746; A61B 2017/568

(57) ABSTRACT

Systems, devices, and methods are described for orthopedic guides. In certain embodiments, an orthopedic guide includes a first surface structured to fit within an implant, a sleeve component coupled to a second surface of the guide, and an alignment structure having a contour with predetermined surface characteristics that correspond to respective characteristics of a patient's bony anatomy and thereby aligns the guide.

### CROSS REFERENCE TO RELATED APPLICATIONS

This application claims the benefit of U.S. Provisional Patent Application No. 61/733,737, filed Dec. 5, 2012, which is hereby incorporated by reference herein in its entirety.

### BACKGROUND

Surgeons use a variety of surgical instruments when performing a hip arthroplasty to implant a prosthesis such as an acetabular cup into a patient's acetabulum. For example, the surgeon typically uses a reamer or other cutting device to ream the acetabulum to form a socket within which the acetabular cup can be implanted. An impactor may then be used to drive the acetabular cup into place within the acetabulum. When operating, in many instances it will be important for the surgeon to position and orient the surgical instruments as precisely as possible, so that the acetabular cup will ultimately be positioned and oriented as intended. Otherwise, if the acetabular cup is not properly positioned and oriented (for example, if the acetabular cup has too shallow or too high of a cup inclination angle), the patient may experience excessive wear on the acetabular cup, or other components used with the acetabular cup, as well as dislocation, impingement, limited ranges of motion, infection, or rejection of the implant.

### SUMMARY

Disclosed herein are systems, devices, and methods for implanting and aligning orthopedic implants. In certain implementations, the systems, devices, and methods include a guide having a surface that is at least in part patient-matched (e.g., to a particular patient's acetabular rim) such that the guide fits in a preferred position and orientation around the perimeter of the acetabular rim. The guide may be used to align and impact an orthopedic implant (e.g., an acetabular cup) into the patient's anatomy. In certain implementations, there may be provided a series of guides. For example, a first guide may provide a predetermined alignment during reaming and/or implant impaction, and a second guide may provide predetermined screw placement, once the implant is seated into the acetabulum.

According to one aspect, an orthopedic guide comprises a first surface structured to fit within an implant, a sleeve component coupled to a second surface of the guide, and an alignment structure having a contour with predetermined surface characteristics that correspond to respective characteristics of a patient's bony anatomy and thereby aligns the guide. In certain implementations, the sleeve component is hollow and shaped to translate along an insertion device. The sleeve component may include a first portion having a first diameter and a second portion having a second diameter that is different than the first diameter. In certain implementations, the second diameter is greater than the first diameter. The second portion may include a mating feature that mates with a complementary feature in the second surface of the guide. In certain implementations, the sleeve component is permanently affixed to the second surface of the guide. In certain implementations, the orthopedic guide further comprises a sleeve lock component that, when actuated, fixes the relative position of the sleeve along an insertion device.

In certain implementations, the guide has a rim along the first surface, and the alignment structure is coupled to the rim. The alignment structure may include an arm with a first end coupled to the rim of the guide and a second end coupled to the contour. In certain implementations, the orthopedic guide further comprises a keying structure for aligning the guide within the implant. The keying structure may include at least one protrusion on the rim. In certain implementations, the keying structure comprises a tapered portion of the first surface. In certain implementations, the keying structure comprises a protrusion on the first surface structured to fit within a hole of the implant.

According to one aspect, a method for performing at least part of a surgical procedure comprises coupling an insertion guide to the orthopedic implant, wherein the insertion guide has a predetermined configuration that corresponds to a respective anatomic landmark site, aligning the orthopedic implant using a sleeve that is coupled to the insertion guide, and removing the insertion guide from the orthopedic implant. In certain implementations, the method further comprises impacting the orthopedic implant after the aligning. The insertion guide may be removed from the orthopedic implant during the impacting. In certain implementations, the method further comprises translating the insertion guide and the orthopedic implant along an alignment tool, wherein the insertion guide and the sleeve do not rotate relative to one another but are free to rotate with respect to the alignment tool. In certain implementations, the method further comprises, after removing the insertion guide from the orthopedic implant, coupling a fixation guide to the orthopedic implant, wherein the fixation guide has a predetermined configuration that corresponds to a respective anatomic landmark site. In certain implementations, the respective anatomic landmark site of the insertion guide and the fixation guide is the same anatomic landmark. In certain implementations, the method further comprises aligning a guide hole from the fixation guide with an aperture in the implant.

According to one aspect, a kit is provided that comprises a first orthopedic guide comprising a sleeve component, and a second orthopedic guide comprising a plurality of apertures structured to receive a fixation element, wherein each of the first and second orthopedic guides comprises an alignment structure having a contour with predetermined surface characteristics that correspond to respective characteristics of a patient's bony anatomy. In certain implementations, the kit comprises a third orthopedic guide comprising a surface structured to mate with a reaming device.

According to one aspect, an orthopedic guide comprises a first surface structured to fit within an implant, translation means coupled to a second surface of the guide, and means for aligning the guide relative to a patient's bony anatomy, said means comprising a contour with predetermined surface characteristics that correspond to respective characteristics of the bony anatomy. In certain implementations, the translation means is hollow and shaped to translate along an insertion device. The translation means may include a first portion having a first diameter and a second portion having a second diameter that is different than the first diameter. In certain implementations, the second diameter is greater than the first diameter. The second portion may include a mating feature that mates with a complementary feature in the second surface of the guide. In certain implementations, the translation means is permanently affixed to the second surface of the guide. In certain implementations, the orthopedic guide further comprises locking means that, when actuated, fixes the relative position of the translation means along an insertion device.

In certain implementations, the guide has a rim along the first surface, and wherein the means for aligning is coupled to the rim. The means for aligning may include an arm with a first end coupled to the rim of the guide and a second end coupled to the contour. In certain implementations, the orthopedic guide further comprises keying means for aligning the guide within the implant. The keying means may include at least one protrusion on the rim. In certain implementations, the keying means comprises a tapered portion of the first surface. In certain implementations, the keying means comprises a protrusion on the first surface structured to fit within a hole of the implant.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1A shows a schematic cross-sectional view of an illustrative orthopedic guide and implant assembly;

FIG. 1B shows a perspective view of the orthopedic guide of FIG. 1A;

FIG. 2 shows a perspective view of the sleeve component of FIG. 1A according to certain embodiments;

FIG. 3 shows a schematic cross-sectional view of an illustrative orthopedic guide and implant assembly;

FIG. 4 shows a side elevation view of an illustrative alignment/preparation tool and locking sleeve;

FIG. 5 shows various perspective views of an orientation feature for an orthopedic guide and sleeve component;

FIG. 6 shows a side elevation view of an illustrative insertion guide and fixation guide;

FIGS. 7A-7C show an illustrative orthopedic guide and implant assembly at various locations along an alignment/preparation tool;

FIGS. 8A and 8B show an illustrative orthopedic guide and implant assembly at various locations relative to a patient's anatomy;

FIG. 9 shows an illustrative flow chart for planning and executing an orthopedic procedure using patient-matched components;

FIG. 10 schematically illustrates a system for facilitating the steps of the process depicted in FIG. 9; and

FIG. 11 shows an illustrative flow chart for making an orthopedic guide having patient-matched features and various steps in a procedure for using the guide.

### DETAILED DESCRIPTION

To provide an overall understanding of the systems, devices, and methods described herein, certain illustrative embodiments will be described. Although the embodiments and features described herein are specifically described for use in connection with acetabular systems, it will be understood that all the components, connection mechanisms, adjustable systems, manufacturing methods, and other features outlined below may be combined with one another in any suitable manner and may be adapted and applied to medical devices and implants to be used in other surgical procedures, including, but not limited to knee arthroplasty, spine arthroplasty, cranio-maxillofacial surgical procedures, shoulder arthroplasty, as well as foot, ankle, hand, and other extremity procedures.

The following disclosure provides systems, devices, and methods for guides for implanting and aligning orthopedic implants (e.g., an acetabular shell, cup, cage or augment) using a positioner/impactor or other suitable alignment tool or, in certain embodiments, preparing an acetabulum to receive an orthopedic implant using a reamer or other suitable preparation tool. The guide may include at least one position indicator with a patient-matched surface feature that contacts the pelvis near or around the acetabulum and provides a predetermined orientation of the implant (or in certain embodiments, the preparation device) with respect to the anatomical reference frame of the patient, where the guide is removably attachable to the implant (or the preparation tool). The systems, devices, and methods further include a translational feature which permits unilateral translation along a shaft of the alignment tool or preparation tool and, in certain embodiments, include an orientation feature that positions the hemisphere of the orthopedic implant (or preparation tool) to that of the guide in a particular orientation.

The alignment/preparation tool includes a shaft and a feature that removably attaches to the orthopedic implant and/or acetabular reamer. This feature can be a metal rod with an end that screws into the apex hole of the orthopedic implant, or it can be a reamer shaft which attaches to the reamer or other acetabular preparation device. In a preferred embodiment, the predetermined orientation of the implant or reamer/preparation device tool determines the inclination (also called “abduction”) and anteversion angles with respect to the patient's anatomical axis.

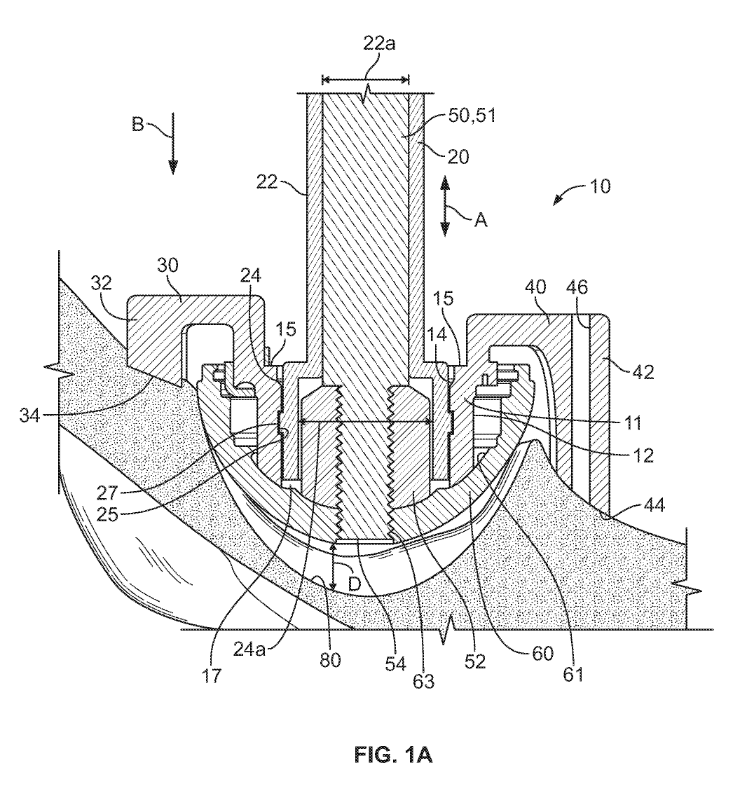

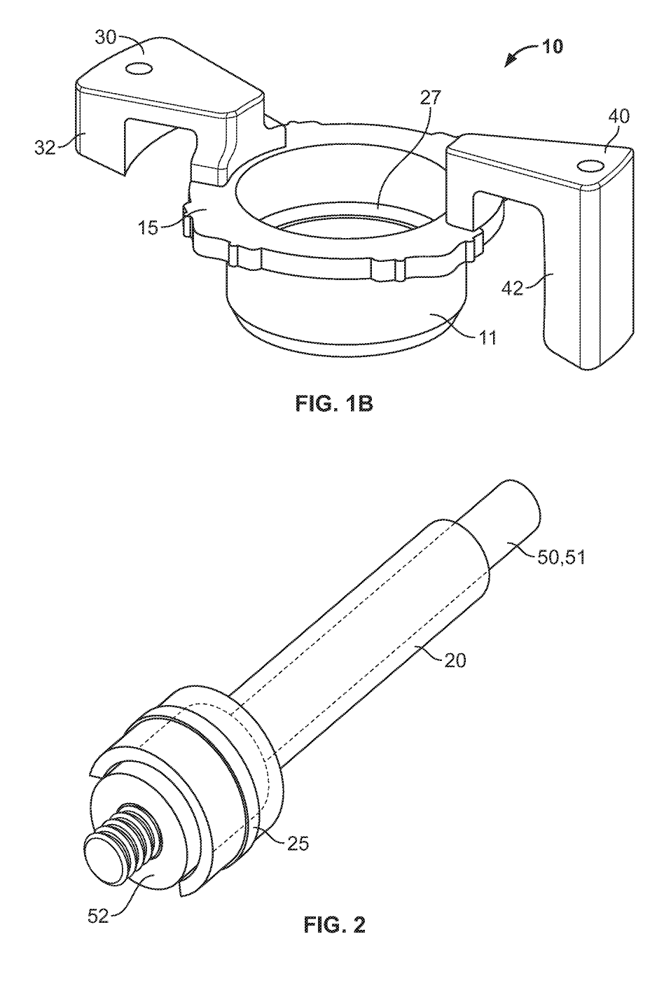

FIGS. 1A and 1B show an orthopedic guide 10 according to certain embodiments.

The orthopedic guide 10 includes a cup portion 11 having an outer surface 12 that is structured to mate with an implant 60 (e.g., an acetabular shell, cup, cage, or augment). The outer surface 12 of the orthopedic guide 10 fits within an interior surface 61 of the implant 60. The guide 10 directs placement and alignment of the implant 60. In certain embodiments, the implant 60 is a standard acetabular cup, for which the guide 10 provides customized placement, orientation, and fixation for a specific patient. In certain embodiments, the implant 60 is a customized device based on patient-matched data. In practice, as shown in FIG. 1A, the guide 10 and the implant 60 are configured together by inserting the guide 10 within the implant 60 to guide the placement of the implant 60 into a desired position and orientation within the patient's anatomy. The cup portion 11 of the guide 10 has a substantially hemispherical shape and fits within the implant 60, with the outer surface 12 of the cup portion 11 coupling with an inner surface 61 of the implant 60. When so configured and placed, the implant 60 is positioned to fit next to the applicable anatomy and the guide 10 overlays the implant 60.

The orthopedic guide 10 further includes a rim 15 about an upper portion of the guide 10 to which alignment structures 30, 40 are coupled. The alignment structures 30, 40 are used for placement and alignment of the implant 60 in a predetermined orientation. In certain embodiments, the alignment structure 30 includes an arm 32 with a patient-matched surface 34 structured to form a complementary fit with a specific portion of the patient's anatomy, such as a patient's acetabulum, in a unique orientation to align the implant 60 to the acetabulum as determined in a pre-operative plan based on patient data. As shown in FIG. 1A, there are two alignment structures 30, 40, although it will be understood that in certain embodiments one alignment structure may be used or, in other embodiments, more than two alignment structures may be used. Alignment structure 40 similarly includes an arm 42 that extends in a direction of the patient's anatomy and has a patient-matched surface 44 that contacts the patient's anatomy. Either or both of the alignment structures 30, 40 can include a guide-pin hole 46.

As discussed above, the guide may include a translation feature that allows for translation along a shaft of the alignment/preparation tool. In certain embodiments, the translation feature of the guide may be an aperture within the guide that has a diameter sized to allow the guide to move along the shaft of the alignment/preparation tool. For example, the guide 10 of FIG. 1A includes an aperture 17 sized for the shaft 51 of the impactor 50 to extend therethrough. In certain embodiments, the translation feature is a sleeve component that removably attaches onto the shaft of the insertion/preparation tool and allows for translational movement of the guide as the implant is impacted. In certain embodiments, the sleeve component is removably attached to the guide, although in other embodiments the sleeve component forms a permanent part of the guide.

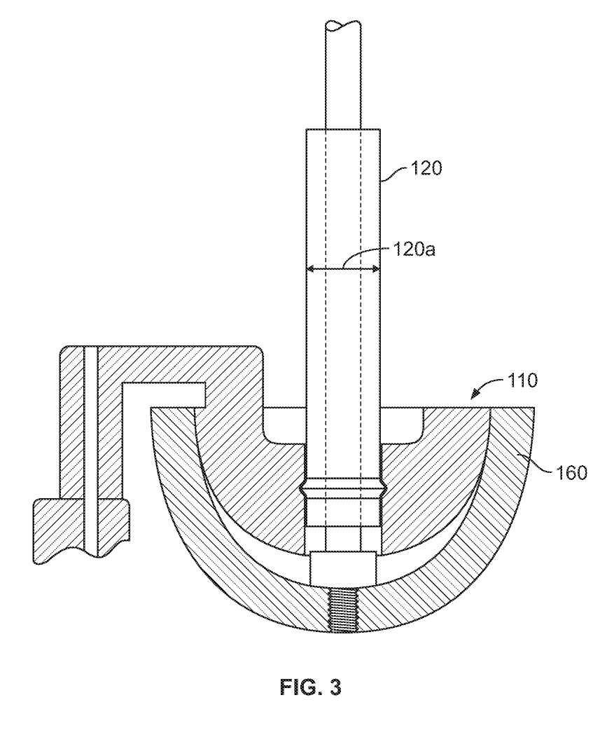

As shown in FIG. 1A, the orthopedic guide 10 has an interior surface 14 that mates with a sleeve component 20. The sleeve component 20 is hollow and structured to slide along the shaft 51 of an impactor 50. In certain embodiments, the sleeve component 20 includes a first portion 22 having a first diameter 22a and a second portion 24 having a second diameter 24a that, as shown in FIG. 1A, is relatively larger than the first diameter 22a. The second portion 24 of the sleeve component 20 is enlarged to fit around an optional attachment 52 that may be coupled to a distal end 54 of the impactor 50. However, it will be appreciated that in certain embodiments the sleeve component 20 may have a substantially uniform diameter along its length. For example, as shown in FIG. 3, the sleeve component 120 has a diameter 120a that is uniform along the length of the sleeve component 120. In such cases, the sleeve component 120 may be structured to seat above the optional attachment piece 52, for example, where the diameter 120a is less than that of the attachment 52, or the sleeve component 120 may have a diameter sufficiently large for the sleeve component 120 to fit around the attachment 52. In still further embodiments, the attachment 52 may not be provided.

The optional attachment 52 may help to prevent scratching of the inner surface 14 of the implant 60, which is typically highly polished to reduce friction with a femoral head. In some embodiments, the attachment 52 may reduce the likelihood that the impactor 50 will jam or otherwise bind to the implant 60 during the procedure (e.g., during alignment or impaction). In some embodiments, the attachment 52 is used to further distribute forces transmitted through the implant 60/impactor 50 connection during the impaction process. The attachment 52 may be secured relative to the impactor 50, the implant 60, or both, in any desired manner, including, but not limited to, threading and/or shoulders on one or both of the impactor shaft 51 and the implant 60, any other suitable coupling mechanism, or any combination thereof. It will be understood that the attachment 52 is merely optional and is not necessary.

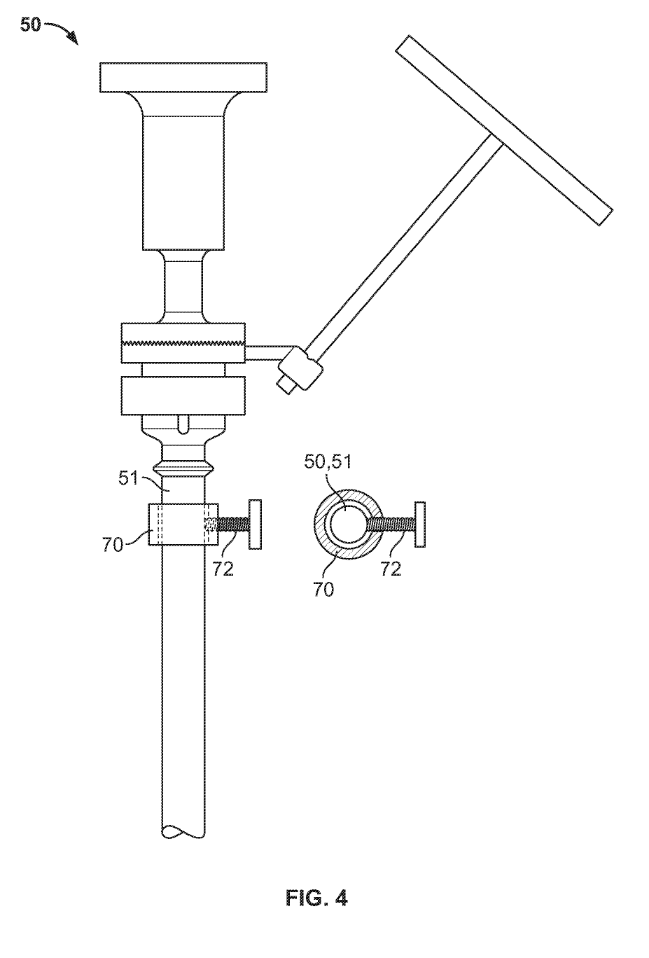

The sleeve component 20 is removably coupled to the inner surface 14 of the guide 10 via a mating feature 25 that is shaped to fit with a complementary feature 27 of the inner surface 14. For example, the enlarged portion 24 of the sleeve component 20 seats inside the guide 10 and may include a male locking detail that affixes to a female locking detail on the inner surface 14 of the cup portion 11. This joins the sleeve component 20 and the guide 10 as one unit. As discussed above, however, in certain embodiments the sleeve component 20 is integrally formed with the orthopedic guide 10 and thus forms a part of the guide 10. The profile of the sleeve component 20 has a “c-shape” for snapping onto the shaft 51 of the impactor 50. For example, FIG. 2 shows a perspective view of the sleeve component 20 if FIG. 1A. As depicted, the sleeve component 20 has a “c-shape” that allows the sleeve component 20 to flex and thereby removably couple with the impactor shaft 51. The sleeve component 20 may be translated using hand pressure or other mechanical means to translate the guide 10 along the axial length of the impactor 50, in the directions of arrow A, until the implant 60 is properly seated. In certain embodiments, a sleeve locking component 70, shown in FIG. 4, may be used to secure the sleeve component 20 in place along the shaft 51 of the impactor 50. The sleeve lock 70 includes a tightening screw 72 that, when tightened, frictionally engages the shaft 51 and the sleeve lock 70 and thereby secures the sleeve lock 70 in place along portion of the shaft 51.

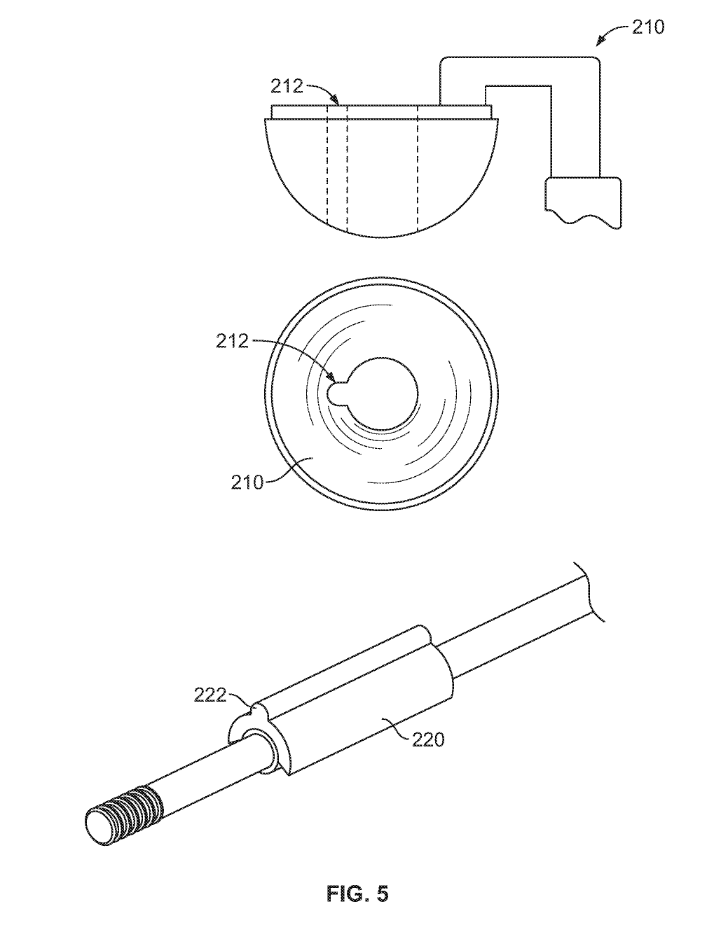

As discussed above, a sleeve component may fix the translation of the guide to an axis of the alignment/preparation tool. In certain embodiments, as shown in FIG. 5, the sleeve component further contains an orientation feature to limit rotation of the guide as the guide translates, for example, when the implant is impacted. The sleeve component 220 has a raised projection 222 along its length that engages and interlocks with a recessed portion 212 of the orthopedic guide 210. The engagement of the projection 222 and the recess 212 aligns the sleeve component 220 with the guide 210 in a predetermined orientation and provides an anti-rotation feature that prevents the guide 210 from rotating relative to the sleeve component 220.

Furthermore, the orthopedic guide and the implant may be coupled in a manner that temporarily affixes the components together. Temporarily locking the guide to the orthopedic implant provides the advantage of allowing placement of the guide and the implant together as an assembled unit. Temporary locking can also prevent axial, rotational, or other movement of the guide relative to the prosthetic cup that would cause misalignment when placing the assembled unit at the surgical site (e.g., the acetabulum). The temporary fixation may be done by one or more temporary fixation structures configured within the implant (e.g., the acetabular cup), the guide, or both. Examples of temporary fixation structures include circumferential bumps that mate with one or more circumferential grooves of an implant, protrusions around the rim that engage and interlock with dimples provided around a circumference of the implant, keying structures such as projections and slots that securely fit together, tapered fittings between the guide and implant, alignment plugs, fins that slide into channels within the implant, and locking pins and pin holes, including the use of split pins. These fixation structures are discussed in detail in PCT Patent Application No. PCT/US2012/040164, filed May 31, 2012, which is hereby incorporated by reference herein in its entirety. Combinations and subcombinations of the locking mechanisms may be used. For example, a guide and cup may be temporarily positioned with one or more of the locking mechanisms described herein. In alternative embodiments, the guide and the prosthetic cup are not locked together. The user holds the guide and cup in position while drilling fastener holes.

In certain embodiments, the orthopedic guide 10 is part of a series of guides. For example, a first guide may provide a predetermined alignment during reaming and/or implant impaction, and a second guide may provide predetermined screw placement, once the implant is seated in the acetabulum. The preferred embodiment for the first guide, also referred to as an “insertion guide,” is shown in FIGS. 1A and 1B. The orthopedic guide 10 is attached, either directly or indirectly (e.g., via an orthopedic implant), to an alignment tool, which can be, for example, an impactor 50 having a shaft 51 that screws into the apex hole or aperture 63 of the implant 60 and is used by the surgeon to orient the implant 60 prior to final insertion by use of the alignment structures 30, 40, which mimic the patient-matched anatomy and provide a predetermined alignment to the patient's anatomical reference frame. Once the proper orientation is determined by the alignment structures 30, 40, the surgeon impacts the alignment tool (e.g., the impactor 50) to firmly seat the implant 60 into the acetabulum and release the guide 10 from the implant 60.

Specifically, upon impaction, the impactor 50 translates in the direction of arrow B by a distance D, which is the distance between the implant 60 and the acetabulum 80. In certain embodiments, the guide 10 is dimensioned such that the patient-matched surfaces 34, 44 make contact with the patient's anatomy (e.g., the acetabular rim) before the body of the implant 60 makes contact with the acetabulum 80, leaving a space D between the implant 60 and the acetabulum 80. In this way, the patient-matched surfaces 34, 44 of the alignment structures 30, 40 may properly align the implant 60 in the desired position and orientation within the acetabulum 80 before impaction. The space D between the implant 60 and the acetabulum 80 prevents, for example, interference between the implant 60 and the patient's acetabulum 80 while the implant 60 is being positioned using the guide 10 (e.g., the implant body does not rub against the acetabulum). In certain embodiments, the impaction force simultaneously seats the implant 60 inside the acetabulum and releases the guide 10 from the implant 60.

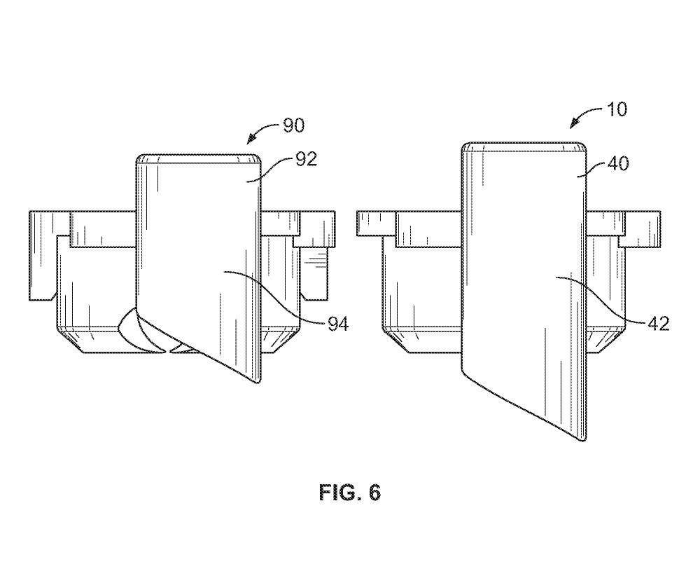

In certain embodiments, the second guide, or “fixation guide,” is attached to the seated implant once the insertion guide is removed. Fixation guides are described in detail in PCT Patent Application No. PCT/US2012/040164, filed May 31, 2012, which is hereby incorporated by reference herein in its entirety. The fixation guide may include various temporary fixation structures, and in certain embodiments the fixation structures used in the insertion guide may be found in the fixation guide. Using similar fixation structures allows for interchangeability between a given implant and the series of guides. The fixation guide may also include alignment structures that mimic the patient-matched anatomy. The alignment structures can reference the same or different parts of the patient anatomy referenced by the insertion guide, however the predetermined placement of the implant with respect to the patient's anatomical reference frame would be the same. The alignment structures of the fixation guide are generally shorter in length than those of the insertion guide because the implant has been seated. For example, FIG. 6 shows an insertion guide 10 and a fixation guide 90 side by side, where the fixation guide 90 has alignment structures 92 with arms 94 that are relatively shorter in length than those of the insertion guide 10. The difference in length between the alignment structures 92 and 40 is approximately the distance D between an unseated implant and the seated implant.

In certain embodiments, an additional guide is provided for preparing the implantation site (e.g., the acetabulum). In such cases, the guide for preparation, for example reaming, would be the first guide used of the series of guides, with the insertion guide being used second and the fixation guide being the third and final guide used.

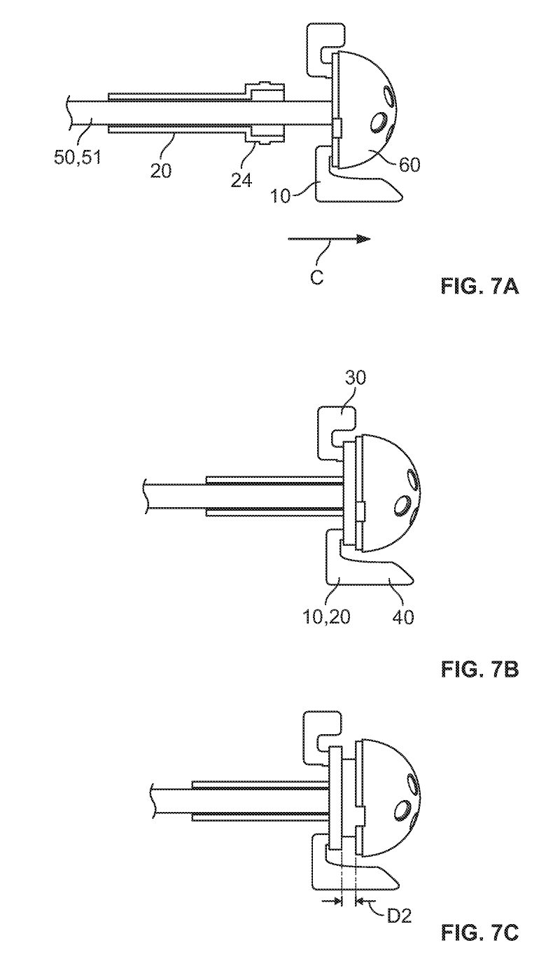

In practice, as depicted in FIGS. 7A-7C, the insertion guide 10 is seated inside the orthopedic implant 60. Fixation structures such as notch location markers may be aligned with a removal notch on the implant. The alignment tool 50 is screwed into the apex 63 of the acetabular implant 60 to join the alignment tool 50 and the acetabular implant 60 as one unit. A sleeve component 20 with an enlarged end 24 toward the acetabular implant 60 is snapped onto the shaft 51 of the alignment tool 50. The sleeve component 20 is then translated along the shaft 51 of the alignment tool 50 in the direction of arrow C and engages the inner surface 14 of the guide 10, thereby joining the sleeve component 20 and the guide 10 into one unit that is coupled to the alignment tool 50 (shown by FIG. 7B). At this point, a surgeon may opt to use a locking sleeve (e.g., the locking sleeve 70 of FIG. 4) to temporarily affix the sleeve/guide unit 10, 20 into position along the shaft 51 of the alignment tool 50. In certain embodiments, the surgeon may opt to manually hold the sleeve/guide unit 10, 20 in place, although the locking sleeve allows the surgeon to free his or her hands while the sleeve/guide unit 10, 20 is fixed in location.

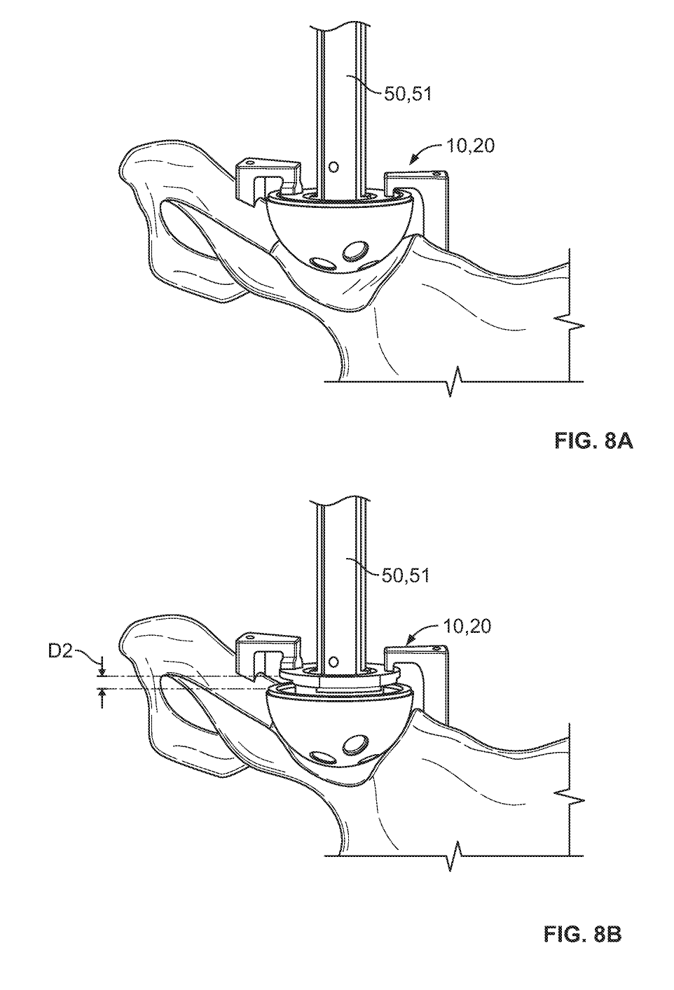

The surgeon places the acetabular implant 60 into the acetabulum using the alignment tool/sleeve/guide assembly. The correct acetabular orientation is found by using the alignment structures 30, 40 and fitting the patient-matched surfaces to specific areas near and around the acetabulum. After the correct orientation is found, the surgeon holds the handle of the alignment tool and releases his or her hold on the sleeve/guide unit 10, 20. The surgeon impacts the implant 60 by striking the end of the device 50 with a mallet or other tool. The alignment structures remain fixed to the patient's anatomy while the acetabular implant seats into the acetabulum. The alignment tool 50 and the implant 60 move in the direction of arrow C during impaction, while the guide 10 remains fixed in place due to contact between the alignment structures and the patient's anatomy. As shown in FIG. 7C, the distance D2 between the guide 10 and the implant 60 is approximately the distance the implant has traveled while being seated into the acetabulum. The relative positions of the implant assembly with respect to a patient's anatomy is shown in FIGS. 8A and 8B.

When the acetabular implant 60 is seated into the acetabulum in the correct orientation, the surgeon unscrews the alignment tool 50 from the apex 63 of the implant 60, removes the first guide 10 and the sleeve 20 and places them aside. In certain embodiments, a second guide secured to the implant 60 using fixation structures such as outer core male tabs that seat into the female locking detail ring inside the implant, with the notch location markers aligned with the removal notch of the implant and alignment structures seated on the patient's anatomy near and around the acetabulum. The alignment structures of the second guide serve as a check for the correct orientation and depth of the acetabular component. If the alignment structures are offset from the patient's anatomy when the second guide is seated into the acetabular implant, the surgeon may opt to screw the alignment tool 50 back into the apex 63 of the acetabular implant and further impact the assembly until the position indicators of the second guide seat flush onto the patient's anatomy and then unscrew the alignment tool from the apex hole of the acetabular component. The surgeon pre-drills screw-holes using patient-specific screw trajectory holes, or may opt to use an existing angle drill guide instrument. The surgeon inserts the screws. The surgeon inserts a liner-removal tool into the removal notch on the acetabular component and removes the second guide. At this point, the acetabular component is fixed in place with screws and is set in the proper orientation for that particular patient.

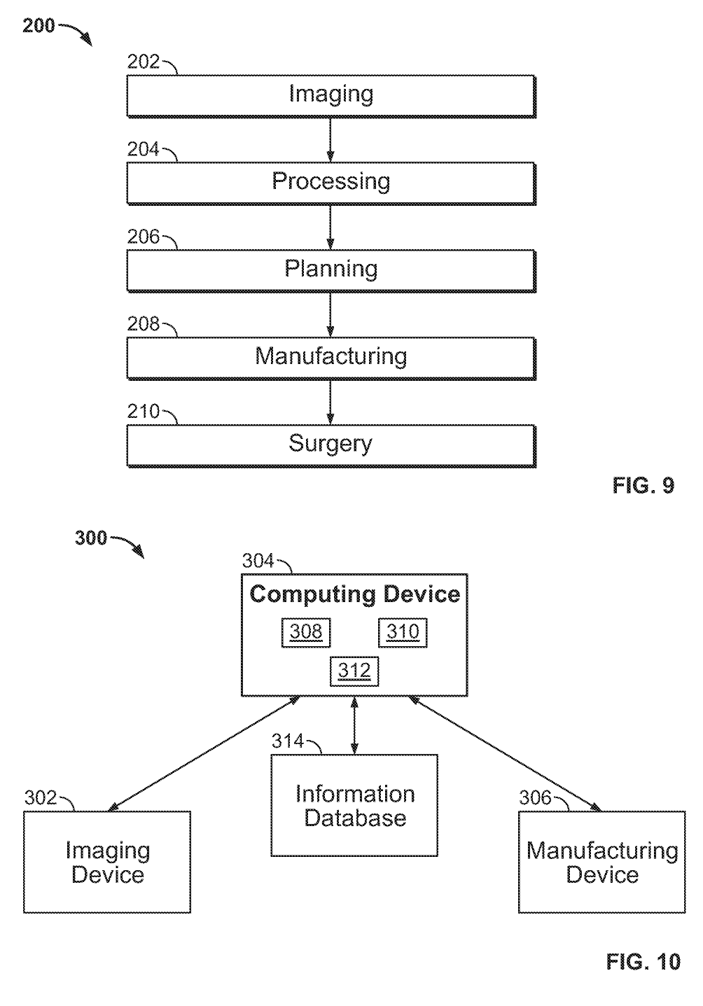

FIG. 9 shows an illustrative flow chart for preoperatively planning and executing an orthopedic procedure using patient-matched components according to certain embodiments. Preferably, the process defines abduction and anteversion angles for the placement of an implant, which, in turn, determines the orientation of the surgical preparation device. For example, the steps of FIG. 9 may be for a procedure on a patient's acetabulum 80. As schematically shown by FIG. 9, the process 200 includes the steps of imaging 202, processing 204, planning 206, manufacturing 208, and performing the surgery 210, although, in some embodiments, at least some of these steps are optional and other steps could be included. A wide variety of systems may be utilized in performing the process 200 shown in FIG. 9. For example, FIG. 10 schematically illustrates a system 300 for facilitating at least some steps of process 200. The system 300 includes an imaging device 302, computing device 304, and manufacturing device 306.

In some embodiments, certain steps of process 200, such as processing 204 and planning 206, may be carried out, wholly or at least partially, using a computing device 304. The computing device 304 may be part of or remote from imaging device or devices 302 used to image the patient and the manufacturing device or devices 306 used to custom manufacture instrumentation, implants or other devices for carrying out the procedure. Computing device 304 may receive or access data reflecting the images of the patient from imaging device 302 through any appropriate communication medium, including, but not limited to, wireline, wireless, optical, magnetic, solid state communication mediums, any other suitable communication medium, or any combination thereof. The computing device 304 represented in FIG. 10 includes a processor 308 that can execute code stored on a computer-readable medium, such as a memory 310. The computing device 304 may be any device that can process data and execute code that is a set of instructions to perform actions. Examples of the computing device 304 include a database server, a web server, desktop personal computer, a laptop personal computer, a server device, a handheld computing device, a mobile device, any other suitable device, or combinations thereof.

In some embodiments, the processor 308 may include a microprocessor, an application-specific integrated circuit (ASIC), a state machine, any other suitable processor, or combinations thereof. The processor 308 may include one processor or any number of processors and may access code stored in the memory 310. The memory 310 may be any non-transitory computer-readable medium capable of tangibly embodying code. The memory 310 may include electronic, magnetic, or optical devices capable of providing processor 308 with executable code. Examples of the memory 310 include random access memory (RAM), read-only memory (ROM), a floppy disk, compact disc, digital video device, magnetic disk, an ASIC, a configured processor, any other suitable storage device, or any combination thereof.

In some embodiments, the computing device 304 may share and/or receive data with additional components through an input/output (I/O) interface 312. The I/O interface 312 may include a USB port, an Ethernet port, a serial bus interface, a parallel bus interface, a wireless connection interface, any other suitable interface capable of allowing data transfers between the computing device and another component, or combinations thereof. The additional components may include components such as an information database 314. In some embodiments, the computing device 304 includes the information database 314.

The patient's anatomy of interest may be imaged using one or more non-invasive imaging technologies, including, but not limited to, computed tomography (CT), magnetic resonance imaging (MRI), X-ray, digital X-ray, ultrasound, any other suitable imaging technology, or any combination thereof. In embodiments using imaging technologies such as CT, MRI, or others, one or more sets of parallel image slices of the patient's anatomy may be obtained, including, for example, a series of transverse slices, sagittal slices, coronal slices, other angulations of slices, or combinations of series thereof. In some embodiments, multiple imaging technologies may be used for the same patient (e.g., X-ray for broader imaging of the overall patient, including other joints, and MRI for the joint of particular interest). The images of the patient's anatomy may, optionally, also include images of existing implants or portions thereof. In some embodiments, non-image based technologies may be used to obtain patient specific information about the patient's anatomy and geometries or other features associated therewith.

Image processing 204 is the next step in the process 200 of FIG. 9, in which at least some of the images may be processed to create an accurate three-dimensional (“3D”) model, other multi-dimensional representation, or other virtual construct representing the geometries and/or selected features of the patient's particular anatomy. In some embodiments, such processing involves segmentation of the images (e.g., separation of at least one set of image slices) to distinguish the anatomy and other structures of interest from the surrounding anatomy and other structures appearing in the image. For example, in certain embodiments, portions of the acetabular rim, including bony or other tissue surfaces associated with the acetabular rim, may be segmented and distinguished from other portions of the images.

In some embodiments, segmentation may be accomplished by manual, automated, or semi-automated processes or any combination thereof. For example, in some embodiments, a technician or other user may (with the assistance of computer assisted design hardware and/or software or other functionality) manually trace the boundary of the anatomy and other structures of interest in each image slice. Alternatively, or additionally, in some embodiments, algorithms or other automated or semi-automated processes could be used to automatically identify the boundaries of interest. In some embodiments, only key points on the anatomy or other structures of interest may be segmented. Processing steps 204 as described above may be used to make a 3D model of the patient's anatomy and other features of interest.

The 3D model or other construct representing the patient's anatomy may be used for pre-surgical planning 206 of the surgical procedure. In some embodiments, pre-surgical planning 206 can include one or more of identifying a desired position and orientation of a implant 60 within the acetabulum, and/or designing a guide 20 comprising a patient-matched surface 34, 44 to conform to portions of an acetabular rim. In various embodiments, the planning 206 may be carried out using manual, semi-automated, or automated functionality.

As described above, the guide 10 may include one or more surfaces 34, 44 that are specifically designed to mimic the patient's particular anatomy (or portions thereof) as determined, for example, by the 3D model of the anatomy. For example, in some embodiments, the patient-matched surface or surfaces 34, 44 can be a negative mold of the patient's anatomy such that the surface 34, 44 uniquely conforms to the patient's anatomy in one particular position and orientation. In other words, the patient-matched surface or surfaces 34, 44 may facilitate achieving a desired position and/or orientation of the guide 10 with respect to the patient's particular anatomy because the patient-matched surface 34, 44 will allow the guide 10 to fully position on the patient's particular anatomy only when the guide 10 is in the desired position and/or orientation.

In some embodiments, the geometries and other aspects of the patient-matched surface 34, 44 are determined in the planning stage 206 by applying a blank (e.g., a wire-frame or similar digital representation) to the 3D model of the patient's anatomy such that the guide 10 is in the desired position and orientation with respect to the patient's anatomy, and then removing from or adding to portions of the blank to create the patient-matched surface 34, 44 conforming to the surface of the patient's anatomy. In some embodiments, other processes performed during the planning stage 206 determine, at least partially or wholly, the position and/or orientation of the blank relative to the 3D model of the patient's anatomy. For example, during planning 206 the position and orientation of the implant 60 may be defined with respect to the patient's acetabulum 80. The planned position of the implant 60 may be used, in combination with the 3D model of the patient's anatomy or the blank, to define the particular shape and other attributes of the guide 10.

Once designed, the guide 10 may be manufactured (step 208 in process 200) using any number of known technologies, including, but not limited to, selective laser sintering, 3D printing, stereo-lithography, other rapid production or custom manufacturing technologies, or any combination thereof. In some embodiments, the manufacturing devices 306 can be remote from the computing devices 304 involved in the processing 204 and planning 206, and data or other information sufficient to manufacture the patient-matched instruments can be exported from the computing devices 304 to the manufacturing devices 306 in any desirable format.

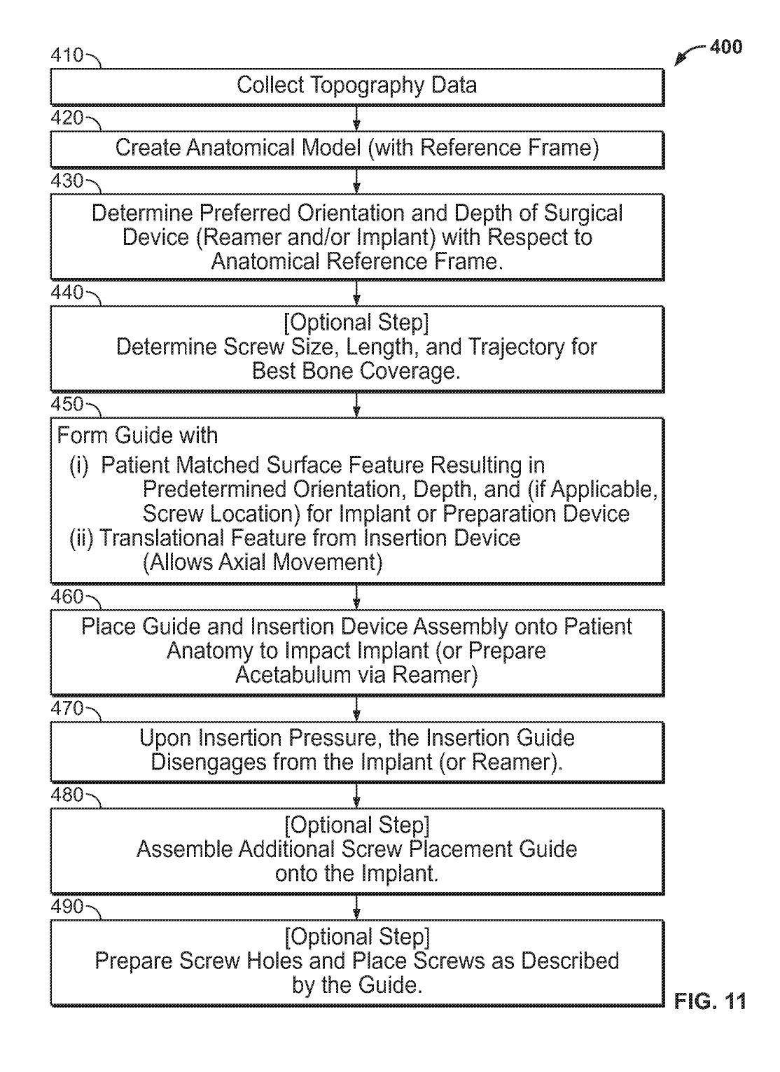

FIG. 11 shows an illustrative flow chart for making an orthopedic guide having patient-matched features and various steps in a procedure for using the guide. In some embodiments, one or more of the steps discussed herein may be performed using stand-alone or networked computer equipment. Such computer equipment, in some embodiments, could include memory, a processor, and input/output features, which facilitate performing at least some of the above identified steps, including creating one or more models. One or more of the above described steps could be performed using a computer assisted design (CAD) software package or other types of design software packages. A wide variety of systems may be utilized in performing the process 400 shown in FIG. 11. For example, the system 300 discussed above with respect to FIG. 10 may facilitate at least some steps of process 400.

The method includes collecting topography data 410, creating an anatomical model 420, and determining a preferred orientation and depth of a surgical device, such as an implant or reamer, with respect to the anatomical reference frame. These steps may be performed using any of the techniques discussed above with respect to the imaging, processing, and planning steps of FIG. 9. An optional step 440 includes determining the screw size, length, and trajectory for best bone coverage. Bone density can serve as a measure of bone quality. In certain forms of imaging data, bone density is proportional to the color density. For example, a higher density image can be indicative of a higher density bone region and suitable areas may show up as sufficiently high density on the imaging data. When judging bone thickness, a user may identify areas that are thick enough to receive the fastener. Generally speaking, a fastener will have better attachment to the bone with the greater number of threads that are passing through the bone. Therefore, the user may align fasteners to be inserted into regions of bone that are thick enough to engage the greatest number of threads on the fastener. Additionally, determining areas of suitable anatomy may include identifying areas that should be avoided, such as blood vessels and nerves. Certain locations may be generally preferable for a fastener. For example, the densest bone in the pelvis is typically located superiorly towards the iliac crest following a posterior thickened ridge, and is a preferred location for screw placement when available.

The location of the holes of the guide may be determined based the areas of suitable anatomy determined in step 420. Specifically, the guide holes are positioned to correspond to the patient's suitable anatomy that is of sufficient quality to accept mechanical fasteners. Additionally, determining the location, position, and orientation of holes and related fasteners may include avoiding sensitive areas such as blood vessels and nerves. The imaging data may provide relevant data to determine unsuitable areas, which should be avoided, as discussed above. Avoidance of critical anatomical features may be accomplished through a user's general surgical and anatomical knowledge. In certain embodiments, determining unsuitable areas to avoid is accomplished in an automated fashion by defining rules of proximity to ensure that fasteners or screws are not spaced too closely. In certain embodiments, no holes are included on the guide. In certain approaches, the holes may not be necessary because the prosthesis or cup may be secured to the patient anatomy without screws. For example, the cup may be secured by a tight fit with thee acetabulum or with bone cement, or the surgeon may decide to place screws without the guide.

There may be any number of holes provided on the prosthesis and the holes may be in any location corresponding to suitable anatomy. In certain embodiments the prosthesis implant is a customized, patient-matched implant. Additionally, patient-matched data can be used to determine the size and other properties of holes and/or fasteners. For example, patient-matched data may be used to determine an appropriately sized fastener to use, and accordingly, an appropriately sized hole in the guide or prosthesis. For example, if there is a particularly large or deep area of suitable anatomy in one location, a larger fastener and a larger hole may be desired for improved fixation. In certain implementations, patient-matched data may be used to determine an appropriate fastener length. In certain implementations, patient-matched data may be used to determine an appropriate fastener type, such as a cortical screw, a cancellous screw, or an osteopenic screw. In certain embodiments, the holes and/or fasteners may be configured to provide for locking of the fastener in the hole in order to increase rigidity of the construct. Such locking features may include, but are not limited to, threads in the hole or on the head of the fastener, deformable materials, geometries creating an interference fit between the fastener and the hole, and any other methodology, mechanism, or structure for locking the fastener in the hole at a fixed angle.

Determining location, position, or orientation of holes and related fasteners may be a manual process or automated. In the case of manual determination, a user makes decisions based on each patient by considering factors such as bone density, thickness, and anatomical structure. For example, a 75-year-old female typically has a different scale of bone density than a 35-year-old male. The user looks at the patient's imaging data as a whole and makes decisions that are an appropriate fit considering all parameters for that patient. Certain locations are generally preferred for fastener placement. For example, the densest bone in the pelvis is typically located superiorly towards the iliac crest following a posterior thickened ridge, and is a preferred location for screw placement when available. In certain embodiments, rules may be applied, to maintain a particular distance (e.g., 10 mm) from areas of concern, (e.g., blood vessel, nerve, or low bone quality).

At step 450, the guide may be formed with a patient-matched surface feature resulting in a predetermined orientation, depth, and, if applicable, screw location for an implant or preparation device. The guide may also be developed having translational features for movement along the alignment or preparation tool (e.g., details for mating with a sleeve component) and/or orientation features for aligning the guide with respect to an implant (e.g., any of the various temporary fixation structures).

Steps 460 through 490 detail various surgical procedures using the guide formed at step 450. These steps include attaching the guide to an implant and alignment tool assembly for aligning and impacting the implant. In certain embodiments, upon application of the insertion pressure, the insertion guide disengages from the implant (or the reamer). Optional steps 480 and 490 include assembling an additional fixation guide (e.g., a screw placement guide) onto the implant, preparing screw holes, and placing screws as defined by the guide.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in acetabular systems, may be applied to systems, devices, and methods to be used in other surgical procedures including, but not limited to, spine arthroplasty, cranio-maxillofacial surgical procedures, knee arthroplasty, shoulder arthroplasty, as well as foot, ankle, hand, and extremities procedures.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application

1. An orthopedic guide comprising:
a first surface structured to fit within an implant;
a sleeve component coupled to a second surface of the guide; and
an alignment structure having a contour with predetermined surface characteristics that correspond to respective characteristics of a patient's bony anatomy and thereby aligns the guide.

2. The orthopedic guide of claim 1, wherein the sleeve component is hollow and shaped to translate along an insertion device.

3. The orthopedic guide of claim 2, wherein the sleeve component comprises a first portion having a first diameter and a second portion having a second diameter that is different than the first diameter.

4. The orthopedic guide of claim 3, wherein the second diameter is greater than the first diameter.

5. The orthopedic guide of claim 3, wherein the second portion has a mating feature that mates with a complementary feature in the second surface of the guide.

6. The orthopedic guide of claim 1, wherein the sleeve component is permanently affixed to the second surface of the guide.

7. The orthopedic guide of claim 1, further comprising a sleeve lock component that, when actuated, fixes the relative position of the sleeve along an insertion device.

8. The orthopedic guide of claim 1, wherein the guide has a rim along the first surface, and wherein the alignment structure is coupled to the rim.

9. The orthopedic guide of claim 8, wherein the alignment structure has an arm with a first end coupled to the rim of the guide and a second end coupled to the contour.

10. The orthopedic guide of claim 8, further comprising a keying structure for aligning the guide within the implant.

11. The orthopedic guide of claim 10, wherein the keying structure comprises at least one protrusion on the rim.

12. The orthopedic guide of claim 10, wherein the keying structure comprises a tapered portion of the first surface.

13. The orthopedic guide of claim 10, wherein the keying structure comprises a protrusion on the first surface structured to fit within a hole of the implant.

14. A method for performing at least part of a surgical procedure, the method comprising:
coupling an insertion guide to the orthopedic implant, wherein the insertion guide has a predetermined configuration that corresponds to a respective anatomic landmark site;
aligning the orthopedic implant using a sleeve that is coupled to the insertion guide; and
removing the insertion guide from the orthopedic implant.

15. The method of claim 14, further comprising impacting the orthopedic implant after the aligning.

16. The method of claim 15, wherein the insertion guide is removed from the orthopedic implant during the impacting.

17. The method of claim 14, further comprising translating the insertion guide and the orthopedic implant along an alignment tool, wherein the insertion guide and the sleeve do not rotate relative to one another but are free to rotate with respect to the alignment tool.

18. The method of claim 14, further comprising:
after removing the insertion guide from the orthopedic implant, coupling a fixation guide to the orthopedic implant, wherein the fixation guide has a predetermined configuration that corresponds to a respective anatomic landmark site.

19. The method of claim 18, wherein the respective anatomic landmark site of the insertion guide and the fixation guide is the same anatomic landmark.

20. The method of claim 18, further comprising aligning a guide hole from the fixation guide with an aperture in the implant.

21. A kit comprising:
a first orthopedic guide comprising a sleeve component; and
a second orthopedic guide comprising a plurality of apertures structured to receive a fixation element;
wherein each of the first and second orthopedic guides comprises an alignment structure having a contour with predetermined surface characteristics that correspond to respective characteristics of a patient's bony anatomy.

22. The kit of claim 21, further comprising:
a third orthopedic guide comprising a surface structured to mate with a reaming device.

23-35. (canceled)
